(19) United States
(12) Patent Application Publication
(10) Pub. No.: US 2026/0364136 A1
(43) Pub. Date: Nov. 19, 2026

(54) USES OF BLACK TOMATO EXTRACT FOR RELIEVING DRY AND UNCOMFORTABLE EYES AND IMPROVING VISUAL FUNCTION

(71) Applicant: TCI CO., LTD., Taipei (TW)

(72) Inventors: YUNG-HSIANG LIN, Taipei (TW); Chung-An Tien, Taipei (TW)

(21) Appl. No.: 19/415,823

(22) Filed: Dec. 11, 2025

Related U.S. Application Data
(60) Provisional application No. 63/803,938, filed on May 12, 2025.

(51) Int. Cl. A61K 36/81; A61K 9/00; A61K 47/02; A61K 47/10; A61P 27/04; A61P 27/10

(52) U.S. Cl. CPC A61K 36/81; A61K 9/009; A61K 47/02; A61K 47/10; A61P 27/04; A61P 27/10; A61K 2236/15; A61K 2236/19; A61K 2236/53

(57) ABSTRACT

The use of a black tomato extract in the preparation of a composition for relieving eye dryness and discomfort and/or improving visual functions in a subject is provided. The composition includes a black tomato extract obtained by extracting black tomatoes (Solanum lycopersicum).

### CROSS-REFERENCES TO RELATED APPLICATIONS

This application claims the benefit of U.S. provisional application Ser. No. 63/803,938, filed on May 12, 2025. The entirety of the above-mentioned patent application is hereby incorporated by references herein and made a part of the specification.

### BACKGROUND

### Technical Field

The present disclosure relates to a black tomato extract, and in particular, to black tomato extract and uses thereof.

### Related Art

Modern people spend extended periods using electronic products, such as computers, smart phones, and tablet devices, thereby leading to a significant increase in daily near work time. Blue light emitted from screens, extended periods of focusing at a fixed distance, and lack of proper rest often cause dry eyes, soreness, blurred vision, and fatigue.

With changes in work and entertainment patterns, eye strain has become a common health issue. In the prior art, methods of adjusting lighting environments, using blue light filtering lenses, or applying hot compress or massage are mostly used for helping relax the eyes. However, there is still room for improvement in terms of relief speed, lasting effect, or ease of use.

In addition, some methods fail to take into account of both visual maintenance and comfort in daily use, thereby making it challenging to sustain good visual conditions over the long term.

### SUMMARY

In some embodiments, use of a black tomato extract in the preparation of a composition for relieving eye dryness and discomfort in a subject, wherein the composition includes the black tomato extract, and the black tomato extract is obtained by extracting black tomatoes (Solanum lycopersicum).

In some embodiments, the eye dryness and discomfort includes eye soreness, eye photophobia, blurred vision, foreign body sensation in the eyes, or a combination thereof.

In some embodiments, use of a black tomato extract in the preparation of a composition for improving visual functions in a subject, where the composition includes the black tomato extract, and the black tomato extract is obtained by extracting black tomatoes (Solanum lycopersicum).

In some embodiments, the improvement of visual functions in the subject is improving visual acuity, ocular accommodation, visual contrast sensitivity, retinal light adaptability, or a combination thereof in the subject.

In some embodiments, the improvement of visual acuity in the subject is improving near visual acuity.

In some embodiments, the improvement of visual acuity in the subject is improving distance visual acuity.

In some embodiments, the improvement of ocular accommodation in the subject is improving the capability of the ciliary muscle in the subject to contract and relax.

In some embodiments, the improvement of visual contrast sensitivity in the subject is improving visual contrast sensitivity in dim environments.

In summary, the black tomato extract of any embodiment of the present disclosure has multiple functions of improving eye functions: the black tomato extract has capabilities of relieving eye dryness and discomfort, such as relieving eye soreness, eye photophobia, blurred vision, and foreign body sensation in the eyes, and has capabilities of improving visual functions in a subject, such as improving visual acuity, ocular accommodation, visual contrast sensitivity, and retinal light adaptability in the subject.

### BRIEF DESCRIPTION OF THE DRAWINGS

FIG. 1 is a line chart showing results of tested near visual acuity of subjects in bright environments at week 0, week 4, and week 8;

FIG. 2 is a line chart showing relative results of tested near visual acuity of subjects in bright environments at week 0, week 4, and week 8;

FIG. 3 is a bar chart showing results of the proportion of subjects in a control group who showed improvement (designated as “improved”) or no improvement (designated as “maintained”) in near visual acuity in bright environments at week 4 and week 8 compared to week 0;

FIG. 4 is a bar chart showing results of the proportion of subjects in an experimental group who showed improvement (designated as “improved”) or no improvement (designated as “maintained”) in near visual acuity in bright environments at week 4 and week 8 compared to week 0;

FIG. 5 is a line chart showing results of tested near visual acuity of subjects in dim environments at week 0, week 4, and week 8;

FIG. 6 is a line chart showing relative results of tested near visual acuity of subjects in dim environments at week 0, week 4, and week 8;

FIG. 7 is a bar chart showing results of the proportion of subjects in a control group who showed improvement (designated as “improved”) or no improvement (designated as “maintained”) in near visual acuity in dim environments at week 4 and week 8 compared to week 0;

FIG. 8 is a bar chart showing results of the proportion of subjects in an experimental group who showed improvement (designated as “improved”) or no improvement (designated as “maintained”) in near visual acuity in dim environments at week 4 and week 8 compared to week 0;

FIG. 9 is a line chart showing results of tested distance visual acuity of subjects in dim environments at week 0, week 4, and week 8;

FIG. 10 is a line chart showing relative results of tested distance visual acuity of subjects in dim environments at week 0, week 4, and week 8;

FIG. 11 is a bar chart showing results of the proportion of subjects in a control group who showed improvement (designated as “improved”) or no improvement (designated as “maintained”) in distance visual acuity in dim environments at week 4 and week 8 compared to week 0;

FIG. 12 is a bar chart showing results of the proportion of subjects in an experimental group who showed improvement (designated as “improved”) or no improvement (designated as “maintained”) in distance visual acuity in dim environments at week 4 and week 8 compared to week 0;

FIG. 13 is a line chart showing results of visual contrast sensitivity of subjects in dim environments and at long distances at week 0, week 4, and week 8;

FIG. 14 is a line chart showing relative results of visual contrast sensitivity of subjects in dim environments and at long distances at week 0, week 4, and week 8;

FIG. 15 is a bar chart showing results of the proportion of subjects in a control group with different levels of visual contrast sensitivity (i.e., good contrast sensitivity, insufficient contrast sensitivity, and poor contrast sensitivity) in dim environments and at long distances at week 0, week 4, and week 8;

FIG. 16 is a bar chart showing results of the proportion of subjects in an experimental group with different levels of visual contrast sensitivity (i.e., good contrast sensitivity, insufficient contrast sensitivity, and poor contrast sensitivity) in dim environments and at long distances at week 0, week 4, and week 8;

FIG. 17 is a line chart showing results of binocular accommodation of subjects at week 0, week 4, and week 8;

FIG. 18 is a line chart showing relative results of binocular accommodation of subjects at week 0, week 4, and week 8; and

FIG. 19 is a bar chart showing the severity of dry eye-related symptoms of subjects in an experimental group at week 0, week 4, and week 8.

### DETAILED DESCRIPTION

As used herein, the term “extract” refers to a product prepared by means of extraction. The extract may be presented in the form of a solution dissolved in a solvent, or may be presented as a concentrate or essence that is free or substantially free of a solvent, or may be presented as dried powder.

Black tomato (Solanum lycopersicum) is a special cultivar bred in recent years. Due to rich anthocyanins in its skin, the black tomato has a dark purple to black appearance. The black tomato has two types of phytochemicals such as anthocyanins and lycopene, has broad-spectrum antioxidant properties, and can effectively scavenge free radicals and reduce oxidative stress.

In some embodiments, the raw material for a black tomato extract is the fruit of black tomato, and the black tomato extract is obtained by subjecting the fruit of black tomato to at least one processing step of chopping, extraction, filtration, concentration, drying and the like. Here, the fruit of black tomato may be washed and then dried, and optionally crushed, cut, coarsely crushed (for example, cut into a size of 12 mm), ground or subjected to other processing steps, so as to obtain raw materials suitable for an extraction procedure.

In some embodiments, the fruit of black tomato is extracted with an extraction solution at a specific temperature for a period of time, so as to obtain crude black tomato extract liquid. The extraction solution is any one selected from water, an acidic solution, an enzyme solution, and an enzyme-containing acidic solution. Moreover, there is no special restriction on the dosage of the extraction solution, as long as it can sufficiently disperse the raw material (e.g., the fruit of black tomato) uniformly therein. By way of example, the volume ratio of the extraction solution to the black tomato is (1 to 20):1 (e.g., 5:1).

In some embodiments, the aforementioned acidic solution is a solution composed of water and one or more acidic components. By way of example, the acidic component may be acetic acid, citric acid, hydrochloric acid, or a combination thereof. In some embodiments, the weight ratio of the acidic component to water is (0.1 to 5):100. By way of example, the acidic component may be citric acid, and the acidic solution is prepared by mixing the citric acid and the water in a weight ratio of (0.5 to 5):100. For example, the acidic solution may be an aqueous solution containing 1% of citric acid by weight.

In some embodiments, the aforementioned enzyme solution is an aqueous solution containing one or more cellulases. By way of example, the cellulase includes endo-beta-glucanase (also referred to as endo-β-glucanase). This cellulase can be used for hydrolyzing (1,3)- or (1,4)-glycosidic bonds in β-D-glucans. By way of example, the cellulase may be a compound cellulase composed of components such as water, sucrose, sodium chloride, endo-β-glucanase (endo-1,3(4)-bond), potassium sorbate. The acidic solution is prepared by mixing the compound cellulase with water at a weight ratio of 0.1:100. For example, the acidic solution may be an aqueous solution containing 0.1% of compound cellulase by weight.

In some embodiments, the enzyme-containing acidic solution is an aqueous solution containing one or more acidic components and one or more cellulases. Here, types of the acidic components and the cellulases are as mentioned above, and thus will not be repeated. By way of example, the extraction solution is an enzyme-containing acidic solution containing 1% of citric acid by weight and 0.1% of compound cellulase (composed of multiple cellulases) by weight.

In some embodiments, the aforementioned “specific temperature” for extraction may be 35 °C to 45 °C (e.g., 40±3 °C). In some embodiments, the aforementioned “period of time” required for extraction is 0.5 h. By way of example, the “period of time” required for extraction is 0.5 h to 2 h. In some embodiments, only after the extraction solution is heated to this specific temperature, black tomatoes are added for extraction for a period of time. Here, since the fruit of black tomato contains heat-sensitive substances, low-temperature extraction is required.

In some embodiments, the aforementioned crude black tomato extract liquid is filtered using a 400-mesh filter screen so as to remove solids, thereby obtaining a black tomato filtrate.

In some embodiments, the aforementioned black tomato filtrate is subjected to vacuum concentration processing so as to obtain black tomato extract liquid. Here, the temperature condition for the vacuum concentration is between 45 °C and 70 °C (e.g., 55±5 °C).

In some embodiments, the aforementioned black tomato liquid is subjected to drying procedure processing so as to obtain black tomato extract powder. Here, the drying procedure may be carried out as freeze drying, spray drying, or the like. By way of example, the black tomato extract liquid is mixed with maltodextrin in a specific ratio, and then spray drying is performed, so as to obtain the solid black tomato extract powder.

Here, the aforementioned black tomato extract liquid and black tomato extract powder are each considered a “black tomato extract” as referred herein. The difference between the two lies only in the form of the substance, with no significant difference in the composition of active ingredients.

Here, unless otherwise specified herein, the term “improvement/relief” herein refer to the optimization or enhancement of normal or suboptimal physiological states; the term “modulation” refers to positive regulation (including induction, stimulation, promotion, and enhancement) or negative regulation (including inhibition, reduction, lowering, and weakening) so as to bring an individual toward the normal state of the described physiological function; the term “individual” refers to a mammal, which may be a human or a non-human animal; and the term “subject” refers to a target upon which the extract, composition, method, or use of the present disclosure acts. The subject may include, but is not limited to, humans, other mammals, animal models, and samples from cells, tissues, or other biological sources. Unless otherwise specified, the term “subject” herein is not limited to a specific type or state, but may also cover in vivo or in vitro subjects.

In some embodiments, the black tomato extract is to relieve eye dryness and discomfort in an individual by functions of reducing eye soreness, relieving eye photophobia, improving blurred vision, and reducing foreign body sensation in the eyes. That is, the visual acuity and eye comfort can be improved after the individual takes the black tomato extract, thus achieving the aforementioned functions.

In some embodiments, the black tomato extract improves a visual function in the aforementioned individual by functions of improving visual acuity, ocular accommodation, visual contrast sensitivity, retinal light adaptability, and the like in the individual. The improvement of visual acuity includes improving near visual acuity and distance visual acuity in different environments (e.g., bright environments and dim environments) The improvement of visual contrast sensitivity includes improving the visual contrast sensitivity in different environments (e.g., the bright environments and the dim environments) and at different distances (e.g., long distances). The improvement of ocular accommodation in the individual includes improving the capability of ciliary muscle in the individual to contract and relax. The improvement of retinal light adaptability includes improving near/distance visual acuity in different environments (e.g., the bright environments and the dim environments) and thus increasing adaptability to external brightness stimuli.

By way of example, after individuals take the black tomato extract, visual acuity can be improved, and eye fatigue caused by blurred vision can be reduced, thereby helping to improve the efficiency in activities such as near reading and work on a computer; after individuals engaged in prolonged near work (e.g., students and office workers) take the black tomato extract, eye fatigue can be relieved, and the probability of blurred vision can be lowered; and/or, after the individuals take the black tomato extract, the error rate of fine motor movements in dim environments can be reduced, and night visual acuity can be improved, thereby meeting the visual needs of night activities (for example: making it easier to see the trajectory of a baseball during a baseball game at night), and meet the needs of darkroom workers (such as darkroom operators, X-ray operators, astronomers, movie players, etc.); and/or, after the individuals take the black tomato extract, night visual acuity can be reduced, glare caused by streetlights and headlights in dim environments can be reduced, and then the movement of pedestrians and vehicles on dim streets can be seen clearly, which helps to improve driving safety (for example, making out road surfaces when walking in the dim environment to avoid falls, improving the driving safety at night, etc.).

In some embodiments, the aforementioned term “near” means that a distance between an individual and an observed object is not more than 40 cm (e.g., 40 cm). In some embodiments, the aforementioned term “distance” means that a distance between an individual and an observed object is not less than 6 m (e.g., 6 m).

In some embodiments, the aforementioned term “bright environment” refers to that illuminance of an environment where an individual is located is about 300 lux to 1000 lux (e.g., 1000 lux). In some embodiments, the aforementioned term “dim environment” refers to that illuminance of an environment where an individual is located is about 1 lux to 50 lux (e.g., 25 lux).

In some embodiments, any one of the aforementioned compositions may be an edible product for non-medical use. In other words, the edible product includes a specific content of black tomato extract. In some embodiments, the edible product may be a general food, a health-care food, or a dietary supplement.

In some embodiments, the aforementioned edible product may be manufactured by using a technology well known to those skilled in this art into a dosage form suitable for oral administration. In some embodiments, the aforementioned general food may be an edible product itself. In some embodiments, the general food may be, but is not limited to, beverages, fermented foods, bakery products, or seasonings.

In some embodiments, the resulting black tomato extract may be further used as a food additive, so as to prepare a food composition containing the black tomato extract. Here, the black tomato extract in any embodiment may be added during the preparation of raw materials by means of a conventional method, or the black tomato extract in any embodiment may be added in a food production process, and is formulated with any edible material into an edible product (i.e., food composition) for human and non-human animal consumption.

In some embodiments, the black tomato extract contained in the aforementioned composition containing the black tomato extract may be liquid or solid. By way of example, the solid may be powder or troche.

In some embodiments, the dosage of the composition is 0.8 g/day of solid black tomato extract (e.g., black tomato extract powder).

### Example 1: Method for Preparing Black Tomato Extract

### Example 1-1: Method for Preparing Liquid Black Tomato Extract (i.e., Black Tomato Extract Liquid)

Firstly, the fruit of black tomato (origin: Gaotai County, Zhangye City, Gansu Province) was roughly crushed into 12 mm fragments (that is, black tomato fragments), and an enzyme-containing acidic solution containing 0.1% of compound cellulase by weight (purchased from Novozymes, commodity: Viscozyme® L, including endo-β-glucanase) and 1% of citric acid by weight (Brand: Weifang Yingxuan Industrial Co., Ltd.) was prepared as an extraction solution. Here, the concentration of the compound cellulase and the concentration of the citric acid were each obtained by conversion based on the extraction solution (that is, amount of liquid inputted in the production process of the black tomato extract), and the weight ratio of the extraction solution to the black tomato fragments was 5:1. Next, the extraction solution was heated to 40 °C±3 °C, and then the black tomato fragments were added, and reflux pump operating pressure was set to 3.0±0.5 kg/cm², and a stable flow was maintained for 30 min of extraction, so as to obtain crude black tomato extract liquid.

Next, the aforementioned crude black tomato extract liquid was filtered with a 400-mesh filter screen to obtain a black tomato filtrate. Here, the soluble solid content (Brix) of the black tomato filtrate at 20 °C was about 1.4±0.3.

Then, the black tomato filtrate was subjected to vacuum concentration using a concentrator (Brand: Yenchen Machinery Co., Ltd., Model: YC-VC-500) at 40 °C+5 °C, and the black tomato filtrate after the vacuum concentration was filtered with the 400-mesh filter screen for the second time, so as to obtain the “black tomato extract liquid”. Here, the soluble solid content (Brix) of the black tomato extract liquid at 20 °C was about 10.0±0.3.

### Example 1-2: Method for Preparing Solid Black Tomato Extract (i.e., Black Tomato Extract Powder)

The black tomato extract liquid prepared in Example 1-1 was used for the subsequent preparation of black tomato extract powder.

Soluble solids in the black tomato extract liquid prepared in Example 1-1 were detected to ensure that every 100 g of black tomato extract liquid contained about 10 g of soluble solids, and maltodextrin was added. Here, the weight ratio of the soluble solids to the maltodextrin was 1:2.

Spray drying was performed on the mixture of the aforementioned soluble solids and maltodextrin, and sieving was performed with a 40-mesh filter screen, so as to obtain the “black tomato extract powder”.

### Example 2: Human Trials

Here, the black tomato extract powder prepared in Example 1-2 was made into test samples, with each sachet of test samples containing 400 mg of solid black tomato extract. Moreover, the test dose involved taking a sachet of test samples in each of the morning and evening, with a total of 800 mg of black tomato extract taken daily.

### Formula of Test Sample:

Each sachet contained 400 mg of black tomato extract powder (as prepared in Example 1-2), 1537 mg of isomaltitol, 20 mg of berry flavoring, 3 mg of sucralose, and 40 mg of calcium phosphate.

### Subjects:

56 subjects, who were adults aged 20 to 35 with independent decision-making capacity. Moreover, all of the subjects met the following conditions:

Visual acuity: Degree of myopia was −5.00 diopters to −9.00 diopters; and the best corrected visual acuity was greater than 0.8. Moreover, none of them had undergone any relevant eye surgery (e.g., strabismus surgery, refractive surgery, etc.).

Physical quality: The body mass index (BMI) value was between 22 and 35.

Lifestyle habits: The time to use a 3C product every day was longer than 6 h.

Other conditions: None of the subjects suffered from systemic diseases affecting the anterior surface of eyes, nor were they on long-term medication affecting the anterior surface of eyes.

### Test Method:

A double-blind, randomized, placebo-controlled trial was conducted, in which a total of 56 subjects were randomly assigned in a subsequent experiment: 28 subjects to a placebo group (control group) and 28 subjects to a black tomato extract group (experimental group). The subjects took one sachet of test sample or placebo in each of the morning and evening daily for 8 consecutive weeks. Eye-related tests were performed at week 0 (i.e., before taking), week 4 (i.e., after taking for 4 weeks), and week 8 (i.e., after taking for 8 weeks) of the trail.

Here, the formula of the placebo was as follows: each sachet contained 60 mg of citric acid, 1577 mg of isomaltitol, 20 mg of berry flavoring, 3 mg of sucralose, and 40 mg of calcium phosphate.

### Test Items:

A. Assessment of Functional Visual Acuity: Testing at long distances (a subject and an observed object were 6 m apart) and near distances (the subject and the observed object were 40 cm apart) in both bright environments (illuminance of 1000 lux) and dim environments (illuminance of 25 lux).

B. Assessment of Functional Visual Contrast Sensitivity: Test at long distances (a subject and an observed object were 6 m apart) in dim environments (illuminance of 25 lux).

C. Binocular Accommodation (total ocular accommodation amplitude).

D. Assessment of Dry Eye: Fill in the questionnaire at week 0, week 2, week 4, week 6, and week 8.

### Example 2-1: Assessment of Functional Visual Acuity

Testing method: Snellen chart and reading-type visual acuity testing tool.

Testing procedure: All subjects participated in testing of visual acuity without changing original spectacle lens power. Distance visual acuity was tested using the wall-mounted Snellen chart at a standard distance of 6 m, while near visual acuity was tested using the reading-type visual acuity testing tool at a distance of 40 cm. The tests were conducted under two lighting conditions respectively: the illuminance in bright environments was 1000 lux, while the illuminance in dim environments was 25 lux. A visual acuity value obtained with the Snellen chart was converted to a logarithm of the Minimum Angle of Resolution (Log MAR) visual acuity value, and then the Log MAR visual acuity value was analyzed. The near visual acuity is represented by a positive Log MAR value. The smaller the value (for example, the closer to 0), the better the visual acuity, and the larger the value, the worse the visual acuity. The distance visual acuity is represented by a negative Log MAR value. The smaller the value (the larger the absolute number), the better the visual acuity, and the larger the value, the worse the visual acuity.

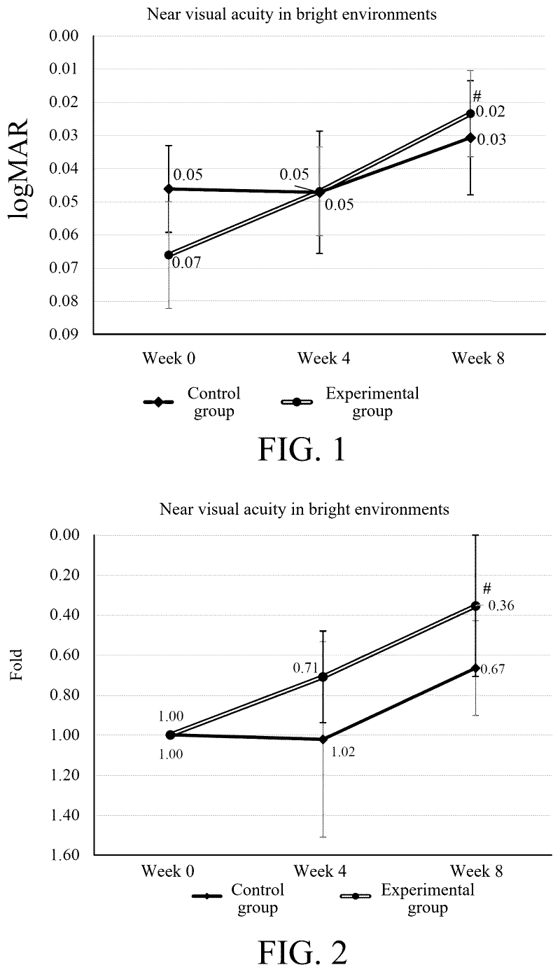

In an experiment of the assessment of functional visual acuity, the near visual acuity and distance visual acuity were assessed in bright environments and dim environments respectively. The results are shown in FIG. 1 to FIG. 12. In the figures, “*” or “#” represents that a p value is less than 0.05. “*” indicates a difference (a difference between different groups at the same time point) between an experimental group and a control group within a current week. “#” indicates a difference (a difference in the same group at different time points) in data compared between week 8/week 4 and week 0.

For the near visual acuity in bright environments:

Reference is made to FIG. 1 to FIG. 4. Referring to FIG. 1, the average Log MAR value measured in the experimental group at week 0 was 0.07, while the average Log MAR value measured in the control group was 0.05. The average Log MAR values measured in the experimental group and the control group at week 4 were both 0.05. At this point, the value of the experimental group decreased compared to that at week 0, while the control group remained basically unchanged. The average Log MAR value measured in the experimental group at week 8 was 0.02, which significantly decreased compared to that at week 0 (as shown in FIG. 1, “#” indicates that the p value was less than 0.05), while the average Log MAR value measured in the control group was 0.03, which also decreased compared to that at week 0 but did not show a significant difference. Moreover, the average Log MAR values measured in the experimental group and the control group at week 0 were both regarded as 1, and then based on this, the fold of change in the average Log MAR values measured in each group at week 4 and week 8 relative to week 0 was calculated. It can be seen that the relative fold of the experimental group at week 4 was 0.71, and the relative fold at week 8 was 0.36, where the value at week 8 significantly decreased compared to that at week 0, as shown in FIG. 2. That is, as the experimental group continued to take the black tomato extract, the average Log MAR value of the experimental group gradually decreased over time, indicating that the near visual acuity of the subjects in the experimental group was significantly improved in bright environments. The relative fold of the control group was 1.02 at week 4, and the relative fold at week 8 was 0.67. That is, the visual acuity of the control group slightly worsened after taking the placebo for 4 weeks, but the visual acuity at week 8 did not improve significantly compared to that at week 0.

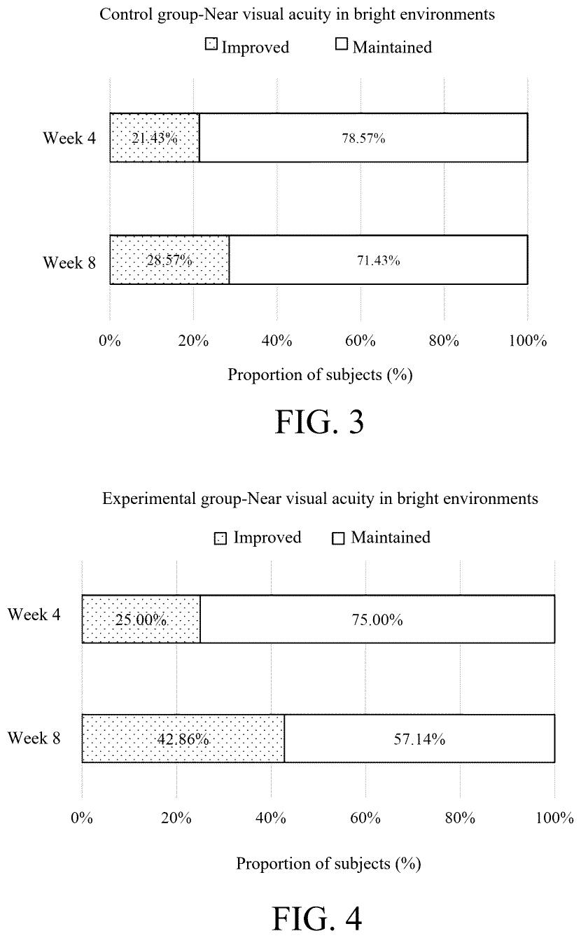

As can be seen from FIG. 3, among the 28 subjects in the control group, the proportion of the number of subjects who felt that their near visual acuity improved in bright environments relative to the total number of the subjects in the control group was 21.43% (about 6 subjects) at week 4 and 28.57% (about 8 subjects) at week 8 (see the group designated as “improved” in FIG. 3), while the proportion of the number of subjects who felt that their visual acuity remained unchanged relative to the total number of the subjects in the control group was 78.57% (about 22 subjects) at week 4 and 71.43% (about 20 subjects) at week 8 (see the group designated as “maintained” in FIG. 3). That is, after taking the placebo for 4 and 8 consecutive weeks, there was only a slight difference in the proportion of subjects who had improvement in their near visual acuity in bright environments.

As can be seen from FIG. 4, among the 28 subjects in the experimental group, the proportion of the number of subjects who felt that their near visual acuity improved in the bright environments relative to the total number of the subjects in the experimental group was 25% (about 7 subjects) at week 4 and 42.86% (about 12 subjects) at week 8 (see the group designated as “improved” in FIG. 4), while the proportion of the number of subjects who felt that their visual acuity remained unchanged relative to the total number of the subjects in the experimental group was 75% (about 21 subjects) at week 4 and 57.14% (about 16 subjects) at week 8 (see the group designated as “maintained” in FIG. 4). After taking the black tomato extract for 4 and 8 consecutive weeks, the proportion of subjects who had improvement in their near visual acuity in the bright environments significantly increased.

Moreover, compared to the control group, the proportion of subjects in the experimental group who had significant improvement in their near visual acuity in the bright environments increased by 14.3% at week 8.

For the near visual acuity in the dim environments:

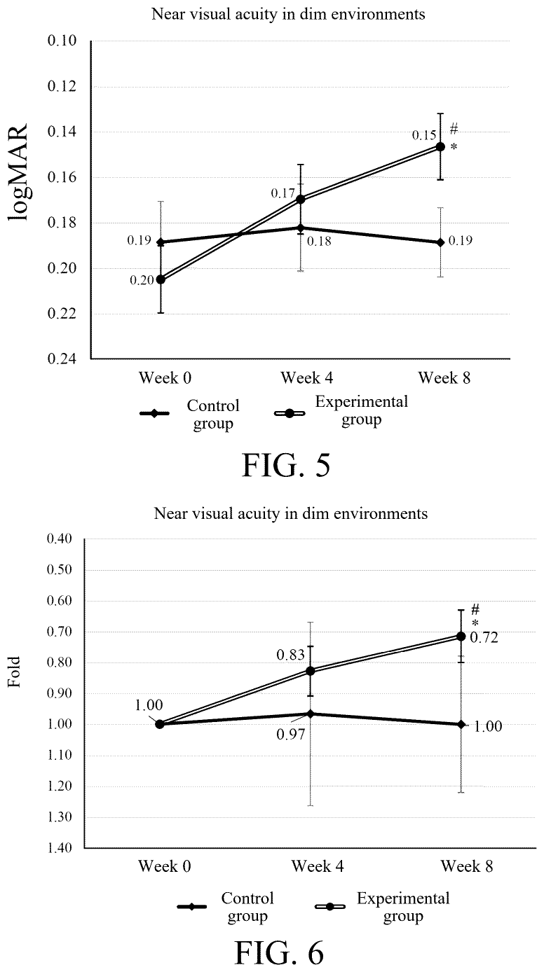

Reference is made to FIG. 5 to FIG. 8. Referring to FIG. 5, the average Log MAR value measured in the experimental group at week 0 was 0.20, while the average Log MAR value measured in the control group was 0.19. The average Log MAR value measured in the experimental group at week 4 was 0.17, while the average Log MAR value measured in the control group was 0.18. The average Log MAR value measured in the experimental group at week 8 was 0.15, which significantly decreased compared to that measured at week 0 and that in the control group at week 8 (as shown in FIG. 5, “#” and “*” indicate that the p value was less than 0.05), while the average Log MAR value measured in the control group was 0.19, which was the same as that measured at week 0. Moreover, the average Log MAR values measured in the experimental group and the control group at week 0 were both regarded as 1, and then based on this, the fold of change in the average Log MAR values measured in each group at week 4 and week 8 relative to week 0 was calculated. The relative fold of the experimental group was 0.83 at week 4, and the relative fold at week 8 was 0.72, as shown in FIG. 6. That is, as the experimental group continued to take the black tomato extract, the average Log MAR value of the experimental group gradually decreased over time, indicating that the near visual acuity of the subjects in the experimental group was significantly improved in the dim environments. The relative fold of the control group was 0.97 at week 4, and the relative fold at week 8 was 1.00. That is, the average Log MAR value of the control group did not change significantly after taking the placebo for 4 or 8 weeks, indicating that the near visual acuity of the control group did not improve in the dim environments after taking the placebo for 4 or 8 weeks.

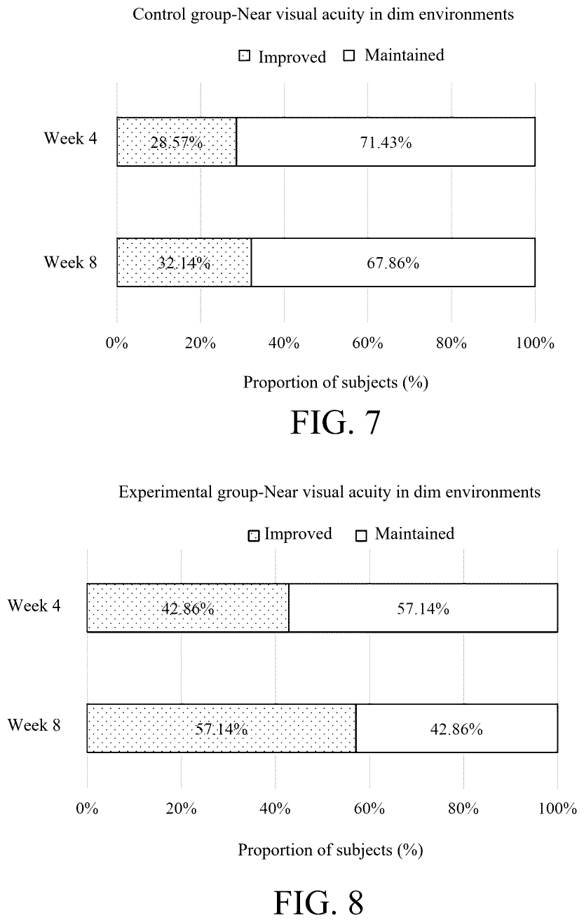

As can be seen from FIG. 7, among the 28 subjects in the control group, the proportion of the number of subjects who felt that their near visual acuity improved in the dim environments relative to the total number of the subjects in the control group was 28.57% (about 8 subjects) at week 4 and 32.14% (about 9 subjects) at week 8 (see the group designated as “improved” in FIG. 7), while the proportion of the number of subjects who felt that their visual acuity remained unchanged relative to the total number of the subjects in the control group was 71.43% (about 20 subjects) at week 4 and 67.86% (about 19 subjects) at week 8 (see the group designated as “maintained” in FIG. 7). That is, after taking the placebo for 4 and 8 consecutive weeks, there was only a slight difference in the proportion of subjects who had improvement in their near visual acuity in the dim environments.

As can be seen from FIG. 8, among the 28 subjects in the experimental group, the proportion of the number of subjects who felt that their near visual acuity improved in the dim environments relative to the total number of the subjects in the experimental group was 42.86% (about 12 subjects) at week 4 and 57.14% (about 16 subjects) at week 8 (see the group designated as “improved” in FIG. 8), while the proportion of the number of subjects who felt that their visual acuity remained unchanged relative to the total number of the subjects in the experimental group was 57.14% (about 16 subjects) at week 4 and 42.86% (about 12 subjects) at week 8 (see the group designated as “maintained” in FIG. 8). After taking the black tomato extract for 4 and 8 consecutive weeks, the proportion of subjects who had improvement in their near visual acuity in the dim environments significantly increased.

Moreover, compared to the control group, the proportion of subjects in the experimental group who had significant improvement in their near visual acuity in the dim environments increased by 25.0% at week 8.

For the distance visual acuity in the dim environments:

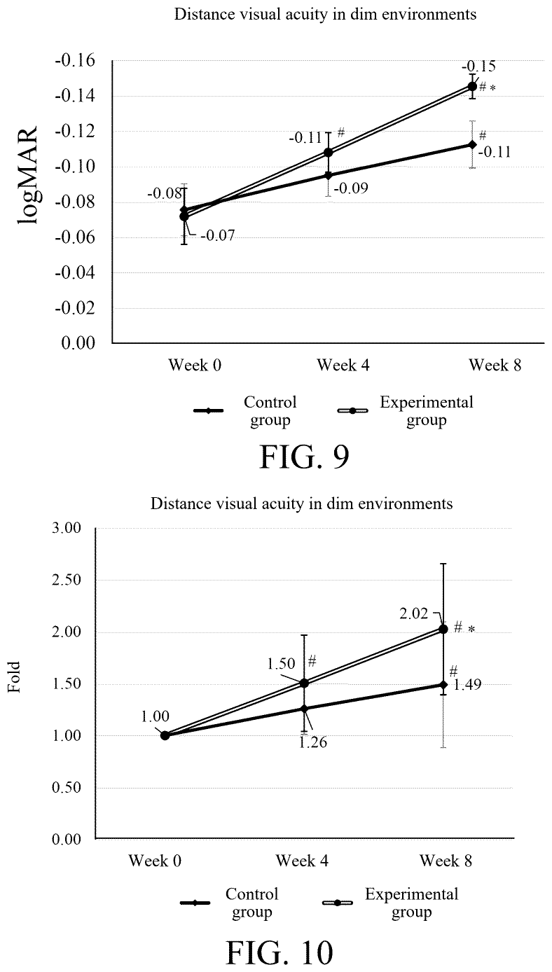

Reference is made to FIG. 9 to FIG. 12. Referring to FIG. 9, the average Log MAR value measured in the experimental group at week 0 was −0.07, while the average Log MAR value measured in the control group was −0.08. The average Log MAR value measured in the experimental group at week 4 was −0.11, which had significantly decreased compared to that measured at week 0, while the average Log MAR value measured in the control group was −0.09. The average Log MAR value measured in the experimental group at week 8 was −0.15, which significantly decreased compared to that measured at week 0 and that in the control group at week 8 (as shown in FIG. 9, “#” and “*” indicate that the p value was less than 0.05), while the average Log MAR value measured in the control group was −0.11. Moreover, the average Log MAR values measured in the experimental group and the control group at week 0 were both regarded as 1, and then based on this, the fold of change in the average Log MAR values measured in each group at week 4 and week 8 relative to week 0 was calculated. Since the distance visual acuity is represented by a negative Log MAR value, the larger the improvement magnitude (i.e., ratio) in proportion, the better the improvement effect. The relative fold of the experimental group was 1.50 at week 4, and the relative fold at week 8 was 2.02, as shown in FIG. 10. That is, as the experimental group continued to take the black tomato extract, the average Log MAR value of the experimental group gradually decreased over time, while the relative ratio increased, indicating that the distance visual acuity of the subjects in the experimental group was significantly improved in the dim environments. The relative fold of the control group was 1.26 at week 4, and the relative fold at week 8 was 1.49. After 8 weeks of intervention, the experimental group had significantly better results than the control group in distance visual acuity tests in the dim environments, indicating that the black tomato extract can significantly improve the distance visual acuity of the subjects in the dim environments compared to the placebo.

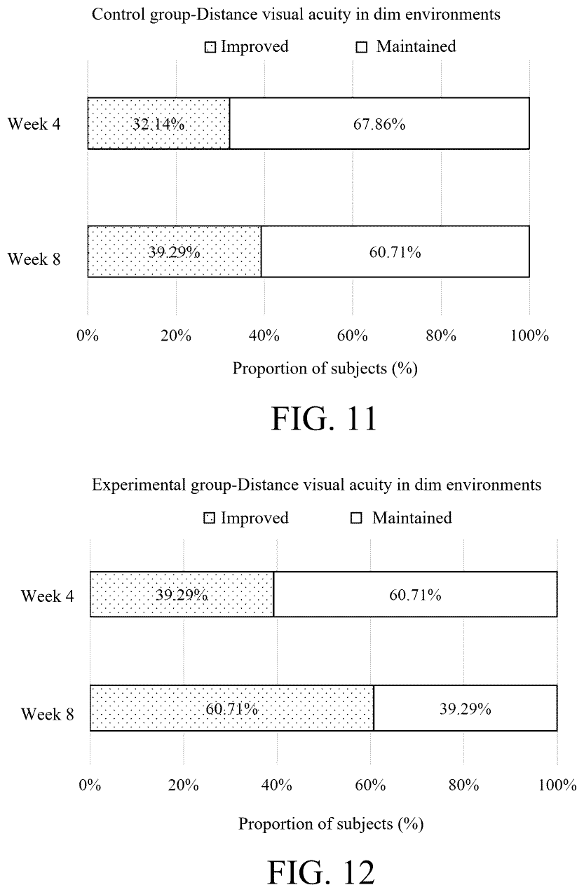

As can be seen from FIG. 11, among the 28 subjects in the control group, the proportion of the number of subjects who felt that their distance visual acuity improved in the dim environments relative to the total number of the subjects in the control group was 32.14% (about 9 subjects) at week 4 and 39.29% (about 11 subjects) at week 8 (see the group designated as “improved” in FIG. 11), while the proportion of the number of subjects who felt that their visual acuity remained unchanged relative to the total number of the subjects in the control group was 67.86% (about 19 subjects) at week 4 and 60.71% (about 17 subjects) at week 8 (see the group designated as “maintained” in FIG. 11). That is, after taking the placebo for 4 and 8 consecutive weeks, there was only a slight difference in the proportion of subjects who had improvement in their distance visual acuity in the dim environments.

As can be seen from FIG. 12, among the 28 subjects in the experimental group, the proportion of the number of subjects who felt that their distance visual acuity improved in the dim environments relative to the total number of the subjects in the experimental group was 39.29% (about 11 subjects) at week 4 and 60.71% (about 17 subjects) at week 8 (see the group designated as “improved” in FIG. 12), while the proportion of the number of subjects who felt that their visual acuity remained unchanged relative to the total number of the subjects in the experimental group was 60.71% (about 17 subjects) at week 4 and 39.29% (about 11 subjects) at week 8 (see the group designated as “maintained” in FIG. 12). After taking the black tomato extract for 4 and 8 consecutive weeks, the proportion of subjects who had improvement in their distance visual acuity in the dim environments significantly increased.

Moreover, compared to the control group, the proportion of subjects in the experimental group who had significant improvement in their distance visual acuity in the dim environments increased by 21.42% at week 8.

It can be seen that taking the black tomato extract helps improve the visual acuity of an individual in different environments and at different distances.

### Example 2-2: Assessment of Functional Visual Contrast Sensitivity

Testing method: Sine-wave grating charts.

Testing procedure: All subjects participated in testing of visual contrast sensitivity without changing original spectacle lens power. Contrast sensitivity refers to the capability to distinguish differences between an object and a background in low-light environments or when images are blurry. The testing of visual contrast sensitivity is different from the testing of the functional visual acuity (Log MAR visual acuity value measurement) in that the visual contrast sensitivity measures the clarity of the smallest legible optotypes. In order to assess visual contrast sensitivity at long distances, a sine wave contrast test was used in this experiment, and testing was conducted in dim environments (illuminance of 25 lux). During the test, the subjects were asked to view the sine-wave grating charts. These charts gradually transitioned from highly clear stripes to blurred ones. Starting from a region with the clearest stripes, the subjects observed until a point where the stripes could no longer be resolved. This point was the limit of their contrast sensitivity. Measured data was presented in contrast sensitivity function curves, and the area under curve (AUC) was further calculated and served as a quantitative index. The larger the area, the better the contrast sensitivity, and an AUC value not less than 30 is considered good contrast sensitivity. An AUC value between 20 and 30 is considered insufficient contrast sensitivity, while an AUC value less than 20 is considered poor contrast sensitivity.

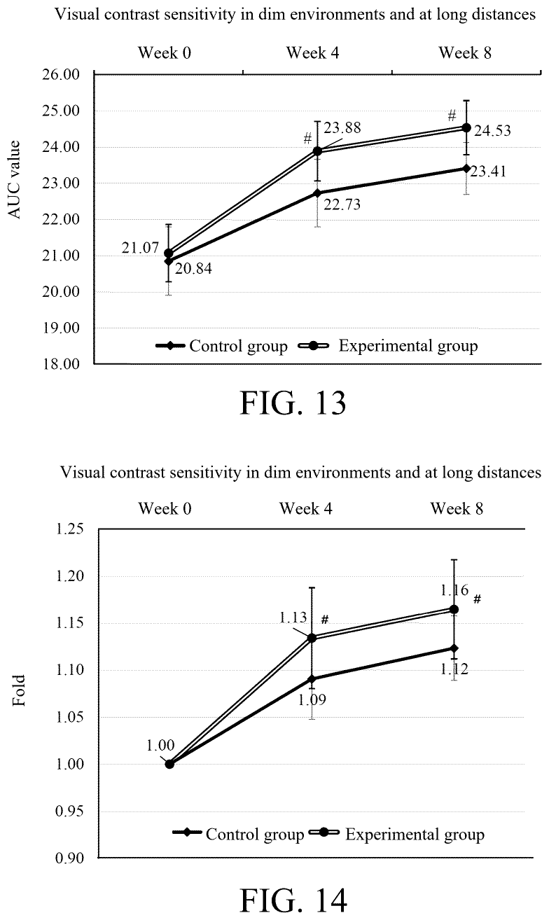

In an experiment of the functional visual contrast sensitivity, the visual contrast sensitivity was tested in dim environments and at long distances. The results are shown in FIG. 13 to FIG. 16. In the figures, “#” represents that a p value is less than 0.05. “#” indicates a difference (a difference in the same group at different time points) in data compared between week 8/week 4 and week 0.

Reference is made to FIG. 13 to FIG. 16. Referring to FIG. 13, the average AUC value measured in the experimental group at week 0 was 21.07, while the average AUC value measured in the control group was 20.84. The average AUC value measured in the experimental group at week 4 was 23.88, which has been significantly increased compared to that measured at week 0 (as shown in FIG. 13, “#” indicates that the p value was less than 0.05), while the average AUC value measured in the control group was 22.73. The average AUC value measured in the experimental group at week 8 was 24.53, which significantly increased compared to that measured at week 0 (as shown in FIG. 13, “#” indicates that the p value was less than 0.05), while the average AUC value measured in the control group was 23.41. The average AUC values measured in the experimental group and the control group at week 0 were both regarded as 1, and then based on this, the fold of change in the average AUC values measured in each group at week 4 and week 8 relative to week 0 was calculated. The relative fold of the experimental group was 1.13 at week 4, and the relative fold at week 8 was 1.16, as shown in FIG. 14. That is, as the experimental group continued to take the black tomato extract, the average AUC value of the experimental group gradually increased over time, indicating that the visual contrast sensitivity of the subjects in the experimental group was significantly improved in the dim environments and at the long distances. The relative fold of the control group was 1.09 at week 4, and the relative fold at week 8 was 1.12. That is, the average AUC value of the control group showed smaller changes after taking the placebo for 4 or 8 weeks, indicating a lower improvement in visual contrast sensitivity in the dim environments and at the long distances.

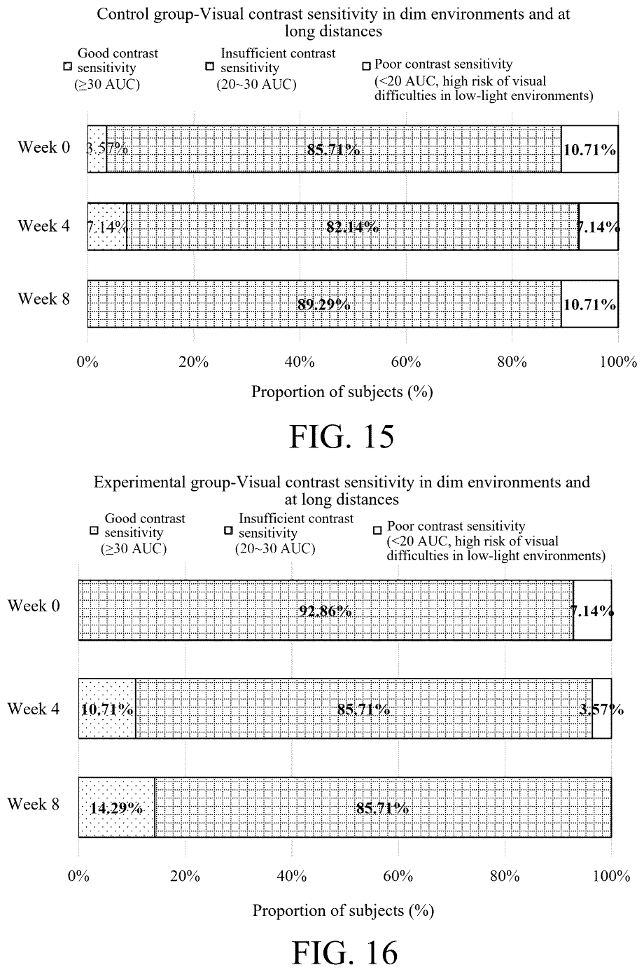

As can be seen from FIG. 15, among the 28 subjects in the control group, the proportion of the number of subjects who felt that they had good visual contrast sensitivity in the dim environments and at the long distances relative to the total number of the subjects in the control group was 3.57% (about 1 subject) at week 0, 7.14% (about 2 subjects) at week 4, and 0% (about 0 subjects) at week 8; the proportion of the number of subjects with insufficient visual contrast sensitivity relative to the total number of the subjects in the control group was 85.71% (about 24 subjects) at week 0, 82.14% (about 23 subjects) at week 4, and 89.29% (about 25 subjects) at week 8; and the proportion of subjects with poor visual contrast sensitivity relative to the total number of the subjects in the control group was 10.71% (about 3 subjects) at week 0, 7.14% (about 2 subjects) at week 4, and 10.71% (about 3 subjects) at week 8. That is, after taking the placebo for 4 and 8 consecutive weeks, the number of the subjects with good visual contrast sensitivity in the dim environments and at the long distances decreased, and most of the subjects believed that their visual contrast sensitivity was insufficient.

As can be seen from FIG. 16, among the 28 subjects in the experimental group, the proportion of the number of subjects who felt that they had good visual contrast sensitivity in the dim environments and the long distances relative to the total number of the subjects in the experimental group was 0% (about 0 subjects) at week 0, 10.71% (about 3 subjects) at week 4, and 14.29% (about 4 subjects) at week 8; the proportion of the number of subjects with insufficient visual contrast sensitivity relative to the total number of the subjects in the experimental group was 92.86% (about 26 subjects) at week 0, 85.71% (about 24 subjects) at week 4, and 85.71% (about 24 subjects) at week 8; and the proportion of subjects with poor visual contrast sensitivity relative to the total number of the subjects in the control group was 7.14% (about 2 subjects) at week 0, 3.57% (about 1 subject) at week 4, and 0% (about 0 subjects) at week 8. That is, after taking the black tomato extract for 4 and 8 consecutive weeks, the number of the subjects with good visual contrast sensitivity in the dim environments and at the long distances increased, and the number of the subjects with poor visual contrast sensitivity decreased to 0.

Moreover, compared to the control group, the proportion of the subjects with good visual contrast sensitivity in the dim environments and at the long distances in the experimental group increased by 14.29% at week 8, while the proportion of the subjects with poor visual contrast sensitivity decreased by 7.14%.

### Example 2-3: Assessment of Binocular Accommodation (Total Ocular Accommodation Amplitude)

Testing method: Negative relative accommodation (NRA) and positive relative accommodation (PRA) were each measured using lenses with diopters of +/−0.25 D.

Testing procedure: When the eyes switch between viewing near and distant objects, the thickness of the lens will be adjusted under the action of the ciliary muscle. This process is called “accommodation”. The overall accommodation of the eyes can be assessed through two key indexes: the negative relative accommodation (hereafter referred to as NRA) and the positive relative accommodation (hereafter referred to as PRA). NRA involves assessing the relaxation accommodation of the eyes when viewing near objects, while PRA involves assessing the capability of the eyes to increase accommodation when focusing. Both of these two measurements were conducted by having subjects view a fixed near reading target. A measurement method for NRA involves adding lenses of +0.25 D stepwise until the subject reports that a visual target becomes blurry, indicating the limit of their relaxation accommodation. The PRA involves adding lenses of −0.25 D stepwise until the visual target becomes blurry, indicating the limit of their focusing capability. These measurements reflect the range of accommodation of the eyes, and the sum of NRA and PRA may indicate the maximum accommodation of the ciliary muscle. Relative accommodation may be calculated by a difference between PRA and NRA (the value of NRA minus that of PRA). The relative accommodation may be used for assessing the overall relaxation and contraction accommodation of the eyes, and the higher the difference, the better the overall accommodation of the eyes.

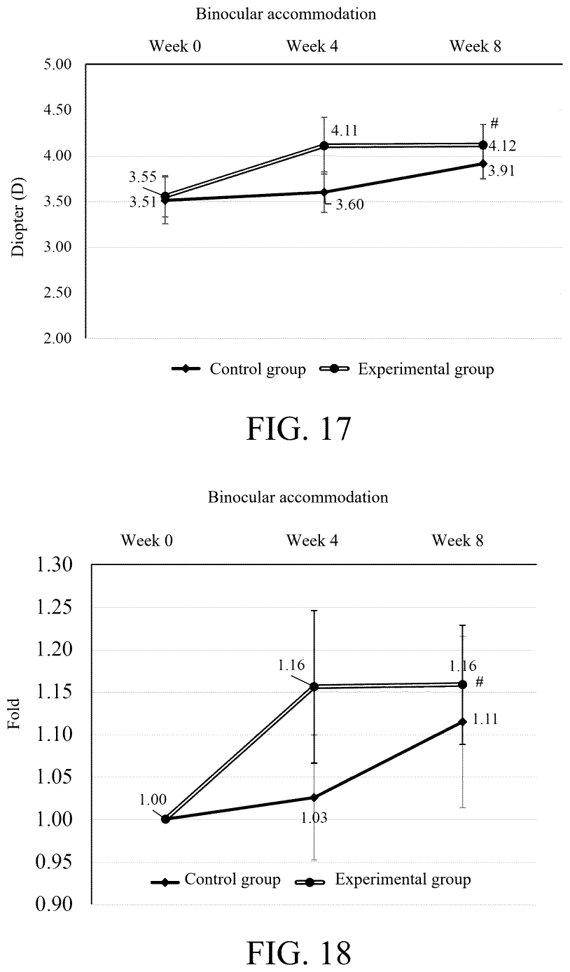

The experimental results of binocular accommodation are as shown in FIG. 17 and FIG. 18. In the figures, “#” represents that a p value is less than 0.05. “#” indicates a difference (a difference in the same group at different time points) in data compared between week 8/week 4 and week 0.

Reference is made to FIG. 17 and FIG. 18. The average relative accommodation (i.e. a difference between PRA and NRA) of the experimental group at week 0 was 3.55 D, while the average relative accommodation of the control group was 3.51 D, with little difference between the two groups. The average relative accommodation of the experimental group at week 4 increased to 4.11 D, while the average relative accommodation of the control group was 3.60 D. The average relative accommodation of the experimental group at week 8 significantly increased to 4.12 D compared to that at week 0 (as shown in FIG. 17, “#” indicated that the p value was less than 0.05), while the average relative accommodation of the control group was 3.91 D. Moreover, the relative accommodation measured in the experimental group and the control group at week 0 was regarded as 1, and then based on this, the fold of change in the relative accommodation measured in each group at week 4 and week 8 relative to week 0 was calculated. The relative fold of the experimental group was 1.16 at week 4, and the relative fold at week 8 was 1.16, as shown in FIG. 18. That is, the relative accommodation of the experimental group increased, indicating that the overall relaxation and contraction accommodation of the eyes of the subjects in the experimental group significantly improved. The relative fold of the control group was 1.03 at week 4, and the relative fold at week 8 was 1.11. That is, the overall relaxation and contraction accommodation of the eyes in the control group did not improve significantly after taking the placebo for 4 weeks.

In addition, relaxing the ciliary muscle can reduce the likelihood of eye soreness, swelling, and even the occurrence of headaches. Therefore, by taking the black tomato extract, the binocular accommodation can be improved, thereby improving the accommodation of the ciliary muscle during relaxation, so that the eye soreness and swelling is relieved, and the headache caused by excessive tension in the ciliary muscle can be avoided.

### Example 2-4: Assessment of Dry Eye

Testing method: The subjects were asked to fill in The Ocular Surface Disease Index (OSDI) before taking (i.e., week 0), week 2, week 4, week 6, and week 8. Reference is made to Table 1 for the OSDI. There are 12 test questions in total, each of which is a single choice. Every option corresponds to a different score (indicated by parentheses in the table).

TABLE 1
Part I. In the past 7 days, have you experienced the following situations?

| | |
|---|---|
| Score | 1. Are your eyes sensitive to light (photophobia)? |
| | All day long (4) |
| | Three quarters of the day (3) |
| | Half of the day (2) |
| | One quarter of the day (1) |
| | Never (0) |
| Score | 2. Do you have a sandy feeling in your eyes (foreign body |
| | sensation) |
| | All day long (4) |
| | Three quarters of the day (3) |
| | Half of the day (2) |
| | One quarter of the day (1) |
| | Never (0) |
| Score | 3. Do you have soreness or discomfort in your eyes? |
| | All day long (4) |
| | Three quarters of the day (3) |
| | Half of the day (2) |
| | One quarter of the day (1) |
| | Never (0) |
| Score | 4. Do you have blurred vision for no apparent reason (Do |
| | things look blurry)? |
| | All day long (4) |
| | Three quarters of the day (3) |
| | Half of the day (2) |
| | One quarter of the day (1) |
| | Never (0) |
| Score | 5. Do you feel that you can't see things clearly, thus affecting |
| | your daily life or work? |
| | All day long (4) |
| | Three quarters of the day (3) |
| | Half of the day (2) |
| | One quarter of the day (1) |
| | Never (0) |

Part II. In the past 7 days, have these following
activities been affected by discomfort in your eyes?

| | |
|---|---|
| Score | 6. Does eye discomfort affect your reading? |
| | Always when reading (4) |
| | Three quarters of the time when reading (3) |
| | Half of the time when reading (2) |
| | One quarter of the time when reading (1) |
| | Never (0) |
| | No experience or activity in this regard (NA) |
| Score | 7. During nighttime movement, has nighttime driving of |
| | vehicles or movement been ever affected by eye discomfort? |
| | Always during nighttime movement (4) |
| | Three quarters of the time during nighttime movement (3) |
| | Half of the time during nighttime movement (2) |
| | One quarter of the time during nighttime movement (1) |
| | Never (0) |
| | No experience or activity in this regard (NA) |
| Score | 8. Have the use of 3C products such as computers or mobile |
| | phones been ever affected by eye discomfort? |
| | Always when using 3C products (4) |
| | Three quarters of the time when using 3C products (3) |
| | Half of the time when using 3C products (2) |
| | One quarter of the time when using 3C products (1) |
| | Never (0) |
| | No experience or activity in this regard (NA) |
| Score | 9. Have watching TV been ever affected by eye discomfort? |
| | Always when watching TV (4) |
| | Three quarters of the time when watching TV (3) |
| | Half of the time when watching TV (2) |
| | One quarter of the time when watching TV (1) |
| | Never (0) |
| | No experience or activity in this regard (NA) |

Part III. In the past 7 days, have your eyes
felt uncomfortable in the following situations?

| | |
|---|---|
| Score | 10. Do your eyes feel uncomfortable when exposed to the wind? |
| | Always uncomfortable when exposed to the wind (4) |
| | Often uncomfortable when exposed to the wind (3) |
| | Sometimes uncomfortable when exposed to the wind (2) |
| | Rarely uncomfortable when exposed to the wind (1) |
| | Never uncomfortable when exposed to the wind (0) |
| | Have never been exposed to the wind (NA) |
| Score | 11. In an air-conditioned (cooled) room, do your eyes feel |
| | uncomfortable? |
| | Always uncomfortable in an air-conditioned room (4) |
| | Often uncomfortable in an air-conditioned room (3) |
| | Sometimes uncomfortable in an air-conditioned room (2) |
| | Rarely uncomfortable in an air-conditioned room (1) |
| | Never uncomfortable in an air-conditioned room (0) |
| | Have never been in an air-conditioned room (NA) |
| Score | 12. In a non-air-conditioned (cooled) room, do your eyes feel |
| | uncomfortable? |
| | Always uncomfortable in a non-air-conditioned room (4) |
| | Often uncomfortable in a non-air-conditioned room (3) |
| | Sometimes uncomfortable in a non-air-conditioned room (2) |
| | Rarely uncomfortable in a non-air-conditioned room (1) |
| | Never uncomfortable in a non-air-conditioned room (0) |
| | Have never been in a non-air-conditioned room (NA) |

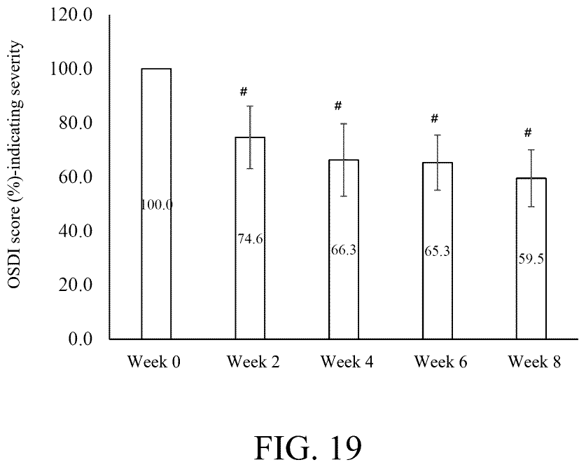

Assessment method for dry eye: OSDI score=(sum of scores)×25/(total number of questions answered). OSDI scores from 0 to 12 indicate “no dry eye, normal”; OSDI scores from 13 to 22 indicate “mild dry eye”; OSDI scores from 23 to 32 indicate “moderate dry eye”; and OSDI scores from 33 to 100 indicate “severe dry eye”. The results are as shown in FIG. 19. In the figures, “#” represents that a p value is less than 0.05. “#” indicates a difference (a difference in the same group at different time points) in data compared with week 0.

Reference is made to FIG. 19. The OSDI score calculated by assessment for the subjects at week 0 was regarded as 100% to represent the severity of dry eye-related symptoms. The severity of the experimental group decreased to 74.6% at week 2, significantly decreased to 66.3% at week 4, significantly decreased to 65.3% at week 6, and significantly decreased to 59.5% at week 8. It can be seen that after the black tomato extract is taken for 2 weeks, symptoms of eye fatigue and dryness are relieved, for example, the severity of dry eye-related symptoms is significantly reduced by 25.4%, eye dryness is effectively relieved, eye photophobia is reduced, blurred vision is relieved, and foreign body sensation in the eyes is relieved, and eye comfort is improved, the visual acuity is improved, and eye discomfort is reduced.

As can be seen from the above, the black tomato extract has the capability to relieve at least one symptom of eye dryness and discomfort, such as eye soreness, eye photophobia, blurred vision, and foreign body sensation in the eyes.

In summary, the black tomato extract according to any embodiment of the present disclosure has various capabilities to relieve eye discomfort and improve visual functions: the black tomato extract has the capability to relieve eye soreness, reduce eye photophobia, relieve blurred vision, reduce foreign body sensation in the eyes, and enhance visual functions in a subject, such as visual acuity, ocular accommodation, visual contrast sensitivity, and retinal light adaptability.

Although the present disclosure has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A use of a black tomato extract in the preparation of a composition for relieving eye dryness and discomfort in a subject, wherein the composition comprises the black tomato extract, and the black tomato extract is obtained by extracting black tomatoes (Solanum lycopersicum).

2. The use according to claim 1, wherein the eye dryness and discomfort comprise eye soreness, eye photophobia, blurred vision, foreign body sensation in the eyes, or a combination thereof.

3. A use of a black tomato extract in the preparation of a composition for improving visual functions in a subject, wherein the composition comprises the black tomato extract, and the black tomato extract is obtained by extracting black tomatoes (Solanum lycopersicum).

4. The use according to claim 3, wherein the improvement of visual functions in the subject is improving visual acuity, ocular accommodation, visual contrast sensitivity, retinal light adaptability, or a combination thereof in the subject.

5. The use according to claim 4, wherein the improvement of visual acuity in the subject is improving near visual acuity.

6. The use according to claim 4, wherein the improvement of visual acuity in the subject is improving distance visual acuity.

7. The use according to claim 4, wherein the improvement of ocular accommodation in the subject is improving the capability of ciliary muscles in the subject to contract and relax.

8. The use according to claim 4, wherein the improvement of visual contrast sensitivity in the subject is improving visual contrast sensitivity in dim environments.
